(19) United States
(12) Patent Application Publication
(10) Pub. No.: US 2015/0359884 A1
(43) Pub. Date: Dec. 17, 2015

(54) MULTI-PHOTON ISOMERISATION OF COMBRETASTATINS AND THEIR USE IN THERAPY

(71) Applicants: Roger H. Bisby, Cheshire (GB); Stanley W. Botchway, Didcot, Oxfordshire (GB); John A. Hadfield, Cheshire (GB); Alan T. McGown, Manchester (GB); Kathrin M. Scherer, Losheim am See (DE)

(72) Inventors: Roger H. Bisby, Cheshire (GB); Stanley W. Botchway, Didcot, Oxfordshire (GB); John A. Hadfield, Cheshire (GB); Alan T. McGown, Manchester (GB); Kathrin M. Scherer, Losheim am See (DE)

(21) Appl. No.: 14/237,642

(22) PCT Filed: Aug. 9, 2012
(86) PCT No.: PCT/GB2012/051942

§ 371 Date: May 13, 2014

(30) Foreign Application Priority Data
Aug. 10, 2011 (GB) 1113815.3

(51) Int. Cl. A61K 41/00; A61N 5/06; C07C 43/23

(52) U.S. Cl. CPC A61K 41/008; C07C 43/23; A61N 5/062

(57) ABSTRACT

Combretastatins and their use in treating conditions characterised by abnormal vasculature or cancer by irradiation of the combretastatin in situ, and kits comprising such compounds.

### FIELD OF THE INVENTION

The present invention relates to combretastatins and their use in therapy, and more particularly to applications of a photochemical isomerisation reaction to convert a relatively inactive trans or E-isomer of a combretastatin to the more active cis or Z-isomer.

### BACKGROUND OF THE INVENTION

The combretastatin group of drugs are a family of substituted stilbenes that are based on the combretastatin A4 molecule first isolated by Pettit and co-workers in 1982 from the African bush willow [1], Combretum caffrum. The most frequently investigated variant, combretastatin A4 (Z-CA4) (and its water soluble prodrug phosphate ester CA4P), is able to act as an anticancer drug by binding strongly to the colchicine site of tubulin and prevent polymerization to functioning microtubules. In the regions surrounding developing tumours this leads to inhibition of angiogenesis through interfering with vascular endothelial-cadherin signalling, thereby depriving the tumour of the nutrients required for growth. In vitro cellular assays of toxicity show CA4 and related molecules to be highly effective and active at nanomolar concentrations in the cell medium. Combretastatins are substituted stilbenes that are two to three orders of magnitude more active as the Z-(cis) isomers (LD50 typically 10⁻⁸M in cellular assays) compared with the corresponding E-(trans) isomers (LD50 in the region of 10⁻⁵M) [12]. This difference in activity is a consequence of the geometrical requirements and substitution pattern required for molecular binding at the colchicine site on the tubulin dimer.

In a previous application WO 02/50007, a range of combretastatin derivatives and prodrugs are described. A process for isomerising the trans or E-isomer of combretastatins to the more active cis or Z-isomer by the action of ultraviolet light in a photoreactor is also disclosed.

There is a continuing need in the art to find effective therapies based on the combretastatin family of molecules, in particular therapies that enable combretastatins to be used in the clinic.

### SUMMARY OF THE INVENTION

Broadly, the present invention is based on the realisation that while known isomerisation reactions for the trans or E-isomer of combretastatins are useful in in vitro studies, it is not generally practical to employ them in therapeutic settings as tissue is largely opaque to ultraviolet (UV) light. This means that in situ conversion, e.g. at the site of a tumour in a patient or in an animal model of cancer, is either not possible or else is not efficient. However, the fact remains that as combretastatins are two to three orders of magnitude more active as the Z-(cis) isomers compared with the corresponding E-(trans) isomers, it would be advantageous if therapies could be based on administering the trans-isomer of a combretastatin as a prodrug to a patient, and converting it to the more active cis-isomer, e.g. at the site of a tumour, thereby localising the cancer cell killing properties of the molecule to the site of the tumour and preferably helping to ameliorate cytotoxicity to normal cells.

When considering this problem, the present inventors realised that in solution at room temperature, E-stilbenes generally have moderate fluorescence quantum yields and sub-nanosecond lifetimes, whereas the Z-isomers are considerably less fluorescent. Both the fluorescence yields and lifetimes of E-isomers increase with solvent viscosity as the competing E→Z isomerization rate decreases. Accordingly, the experiments described herein investigated the electronic excitation of such organic chromophores using two-photon excitation (2PE) because the far red and/or near infra red (NIR) radiation used is capable of penetrating through tissue, and therefore capable of therapeutic application.

Accordingly, in a first aspect, the present invention provides a compound which is a trans-combretastatin, or a derivative, salt, solvate and/or chemically protected form thereof, for use in a method of treating cancer or a condition characterised by abnormal vasculature, wherein the method comprises administering the compound to a patient having cancer or a condition characterised by abnormal vasculature and irradiating the compound with light at a wavelength to isomerise the compound in situ from the trans-isomer to the cis-isomer by a multi-photon process. By way of example, the isomerisation may be carried out by irradiating the trans-isomer of the combretastatin using far red and/or near infra red (NIR) radiation, such as that supplied by a laser.

In a further aspect, the present invention provides a method of treating cancer or a condition characterised by abnormal vasculature, the method comprises administering a compound which is a trans-combretastatin, or a derivative, salt, solvate, and/or chemically protected form thereof, to a patient having cancer and irradiating the compound with light at a wavelength to isomerise the compound in situ from the trans-isomer to the cis-isomer by a multi-photon process. By way of example, the isomerisation may be carried out by irradiating the trans-isomer of the combretastatin using far red and/or near infra red (NIR) radiation, such as that supplied by a laser.

Generally, the combretastatin family of compounds are comprise two aromatic benzene rings, often denoted the A and B rings, linked by an ethene bridge. The A-ring in the structure is generally substituted at the 3, 4, and 5 positions by C₁₋₆ alkyl or C₁₋₆ alkoxy groups, and the B-ring comprises substitutions at the 3 and/or 4-position(s). Optionally, the ethene bridge may additional comprise one or two substitutions in addition to the two benzene rings.

Preferably, the trans-combretastatin used in accordance with the present invention may be represented by Formula I:

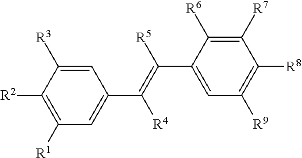

wherein:
R¹, R², and R³ are independently selected from hydrogen, C₁₋₆ alkyl or C₁₋₆ alkoxy, wherein at least one, preferably two, and more preferably all three of R¹, R² and R³ are independently selected from C₁₋₆ alkyl or C₁₋₆ alkoxy;
R⁴ and R⁵ are independently selected from hydrogen, C₁₋₆ alkyl, C₁₋₆ alkoxy, —CO₂H, —CO₂R, —CN, R—C(O)— or —CHO;
R⁶ is selected from hydrogen, hydroxyl, halogen, C₁₋₆ alkyl, C₁₋₆ haloalkyl, C₁₋₆ alkoxy, —O—P(O)(OM⁺)₂ where M+ is a monovalent metal ion, such as sodium or potassium, —NH₂, —NHR, —NRR′, —SR, —NO₂, —CN or —CHO, wherein R and R′ are defined below;
R⁷ is selected from hydrogen, hydroxyl, halogen, —O-ester, —OCO-aryl, —OCO-heteroaryl, —OCO-amino acid, —OCO-peptide, —OCO-polymer, —OCO-sugar, OCO—CHR—NH—BOC, wherein BOC represents a t-butoxycarbonyl group, —O—P(O) (OM⁺)₂ where M+ is a monovalent metal ion such as sodium or potassium, pyridyl ester, —OR, —NO₂, —NH₂, —NHR, —NRR′, —SR, —NHC═OR, —CN or —CHO;
R⁸ is selected from halogen, C₁₋₆ alkyl, C₁₋₆ alkoxy, —NH₂, —NHR, —NRR′, —SR, —CF₃, —CHO, —CN or —C═OR; and
R⁹ is selected from hydrogen, hydroxyl, halogen, C₁₋₆ alkyl, C₁₋₆ haloalkyl, C₁₋₆ alkoxy, hydroxyl, —NH₂, —NHR, —NRR′, —SR, —NO₂, —NH₂, —NHR, —NRR′, —SR, —NHC═OR, —CN or —CHO;
wherein the R and/or R′ substituents, when present, are independently selected from an optionally substituted C₁₋₆ alkyl group;
and salts, solvates, and/or chemically protected forms thereof.

Preferably, the combretastatin compounds represented by Formula I have the following pattern of substituents:

R¹, R² and R³ are independently selected from C₁₋₆ alkyl or C₁₋₆ alkoxy; and/or
R⁴ and R⁵ are independently selected from hydrogen or C₁₋₆ alkyl; and/or
R⁶ is hydrogen; and/or
R⁷ is selected from hydrogen, hydroxyl, halogen, —NO₂, or —NH₂; and/or
R⁸ is selected from C₁₋₆ alkoxy or —N(C₁₋₆ alkyl)₂; and/or
R⁹ is selected from hydrogen, hydroxyl, halogen, —NH₂ or —NO₂;
and salts, solvates, and/or chemically protected forms thereof.

More preferably, the combretastatin compounds represented by Formula I comprise a R⁷ and/or R⁹ halogen substituent which is fluorine.

In any of the compounds used in accordance with the present invention, C₁₋₆ alkyl or C₁₋₆ alkoxy substituents are short chain alkyl or alkoxy groups such methyl, ethyl, methoxy or ethoxy groups.

Particularly preferred compounds represented by Formula I have the following patterns of substituents:

R¹, R² and R³ are methoxy, R⁴, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴, R⁵ and R⁶ are hydrogen, R⁷ is fluorine, R⁸ is methoxy, and R⁹ is hydrogen, and salts, solvates, and/or chemically protected forms thereof; or
R¹, R² and R³ are methoxy, R⁴, R⁵, R⁶ and R⁷ are hydrogen, R⁸ is m-CN, and R⁹ is hydrogen, and salts, solvates, and/or chemically protected forms thereof; or
R¹, R² and R³ are methoxy, R⁴, R⁵ and R⁶ are hydrogen, R⁷ is or —NH₂, R⁸ is methoxy, and R⁹ is hydrogen, and salts, solvates, and/or chemically protected forms thereof; or
R¹, R² and R³ are methoxy, R⁴, R⁵ and R⁶ are hydrogen, R⁷ is hydrogen, R⁸ is —N(methyl)₂, and R⁹ is hydrogen, and salts, solvates, and/or chemically protected forms thereof; or
R¹, R² and R³ are methoxy, R⁴, R⁵ and R⁶ are hydrogen, R⁷ is hydroxyl, R³ is methoxy, and R³ is hydrogen, and salts, solvates, and/or chemically protected forms thereof; or
R¹, R² and R³ are methoxy, R⁴ is C₁₋₆alkyl, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴ is —CN, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴ is —CHO, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴ is —COMe, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴, R⁵ and R⁶ are hydrogen, R⁷ is —O—P(O)(OM⁺)₂ where M+ is a monovalent metal ion, such as sodium or potassium, R⁸ is methoxy, and R⁹ is hydrogen, and salts, solvates, and/or chemically protected forms thereof; or
R¹, R² and R³ are methoxy, R⁴ and R⁵ are hydrogen, R⁶ and R⁷ are hydroxyl, R⁸ is methoxy, and R⁹ is hydrogen, and salts, solvates, and/or chemically protected forms thereof; or
R¹, R² and R³ are methoxy, R⁴ and R⁵ are hydrogen, R⁶ and R⁷ is —O—P(O)(OM⁺)₂ where M+ is a monovalent metal ion, such as sodium or potassium, R⁸ is methoxy, and R⁹ is hydrogen, and salts, solvates, and/or chemically protected forms thereof.

Conveniently, the light for isomerising the combretastatin compounds in the multi-photon process according to the present invention comprises far red and/or near infra red (NIR) radiation as the wavelength of light in this part of the electromagnetic spectrum has an appropriate wavelength for multi-photon absorption by the compound and has the ability to penetrate tissues to a site in a patient where the isomerisation process takes place. In this connection, the multi-photon process is generally a two-photon or a three-photon process, in either case the number of photons being intended to provide approximately the same energy as the single photon used in the corresponding UV isomerisation process. In the case of a two-photon process, 2 photons at a wavelength between 560 nm and 630 nm, and preferably about 600 nm may be used. In the case of the three-photon process, 3 photons at a wavelength between 840 nm and 1200 nm, preferably about 900 nm may be used.

In the present invention, the light for the multi-photon isomerisation is provided by a laser capable of providing high photon densities, preferably in the form of producing sub-picosecond pulses. The powers required are in the range of MW cm⁻² to GW cm⁻² and may be provided in the near infra red by focussing to the diffraction limit the outputs of either a sub-picosecond pulsed titanium sapphire laser (typical average power ca 1-5 mW) or by using a continuous wave near infra red laser, e.g. with a power of ca 100 mW. An example of using lasers in this way is provided in, for example, Konig, Multiphoton Microscopy in Life Sciences. Journal of Microscopy 200 (2): 83-104, 2000. Accordingly, in some embodiments, the present invention may employ a titanium-sapphire laser operating the range of 700-1000 nm with a repetition frequency of 80 MHz and a pulse width of ca 150 femtoseconds or an optical parametric oscillator (OPO) laser, driven by the Ti-sapphire laser providing shorter wavelength region, for example producing wavelengths in the range of 560-630 nm. Conveniently, in the methods disclosed herein comprise the steps of focussing and scanning a laser beam across the area to be illuminated in order to optimise treatment.

The present invention includes the combination of the aspects and preferred features described except where such a combination is clearly impermissible or is stated to be expressly avoided. Embodiments of the present invention will now be described by way of example and not limitation with reference to the accompanying figures.

### BRIEF DESCRIPTION OF THE FIGURES

FIG. 1. Structures of E-combretastatin A4 and analogues used in this study.

FIG. 2. Normalised one-photon absorption spectra of the E combretastatins used in this work. The spectrum of Z-CA4F is shown for comparison with the absorbance at the correct ratio to that of the E-CA4F. Also shown are the 2-photon cross sections for E-CA4 on a wavelength scale (top axis) at twice that used for the one-photon spectra. All spectra were measured in dichloromethane.

FIG. 3. Correlation between fluorescence quantum yield and fluorescence lifetime (both measured at 20° C.) for E-CA4 (♦) and E-CA4F (⋄) in the range of solvents shown in Table 1. Inset: Effect of solvent viscosity on fluorescence lifetimes and quantum yields for the alkanols in Table 1.

FIG. 4. Log-log plots of fluorescence intensity at 400-450 nm from the E-combretastatins (0.5 mM) and the 9-chloroanthracene (10 mM) standard in DCM solutions measured with 620 nm excitation. All the plots have slopes of 2.0±0.2.

FIG. 5. Fluorescence images of CHO cells before (images A1 and B1) and at 30 seconds (A2, B2), 3 minutes (A3, B3) and 6 minutes (A4, B4) after addition of E-CA4F (series A) or E-CA4 (series B) at 22° C. Both combretastatins were added to a final concentration of 10 μmol dm⁻³. Fluorescence at 400 nm was excited by two-photon absorption at 628 nm. Panel C shows the overall average number of counts per pixel () and maximum pixel intensity (□) after addition of E-CA4F (series A).

FIG. 6. Fluorescence lifetime imaging of E-CA4 and E-CA4F in cells and co-localization with Nile Red. A: E-CA4 (100 μmol dm⁻³ in medium) with HeLa cells; cells; B: E-CA4F (50 μmol dm⁻³ in medium) with HeLa cells; C: E-CA4F (50 μmol dm⁻³ in medium) with CHO. Frames show (a) intensity images of combretastatin fluorescence at 400 nm with 628 nm 2-photon excitation, (b) Nile Red fluorescence (515-535 nm) excited at 488 nm, and (c) fluorescence lifetime images. Below are (d) the overlaid images of the drug and Nile Red images (a and b) and (e) the fluorescence lifetime distributions derived from the lifetime images. The bar represents 20 μm.

FIG. 7. Fluorescence intensity (left) and lifetime (middle) images of CHO cells incubated with E-ACA (top) and E-DMAC (bottom) and measured with 2-photon excitation at 628 nm. The frames on the right show the lifetime distribution correspond to the images. Both compounds were added to the cell medium at a final concentration of 10 mol dm⁻³ and the images acquired 10 minutes (E-ACA) and 55 minutes (E-DMAC) after addition. The bar represents 20 μm.

FIG. 8. Comparison of fluorescence spectra of E-CA4 (black curves) and E-DMAC (red curves) in DCM solutions measured using one photon excitation in the Spex fluorimeter (dotted curves) with 2-photon excited spectra (628 nm) of the same compounds after uptake into HeLa cells (full curves).

FIG. 9. Contour plots showing intracellular concentrations of combretastatins in cells at room temperature after incubation for approximately 10 minutes: A: E-CA4 (100 μM added) in HeLa cells; B: E-CA4 (10 μM added) in CHO cells (the image corresponds to that in FIG. 8A); C: E-CA4F (10 μM added) in CHO cells. The concentrations are calculated using the intensity and lifetime at each pixel as described in the text. Each plot represents an area of 70×70 μm. The vertical scales show the combretastatin concentration in μM.

FIG. 10. Structures of the combretastatin analogues used in this work. Photophysical properties including fluorescence quantum yields (c) and fluorescence lifetimes (τ) were measured in DMSO solution.

FIG. 11. Evidence for isomerization of E- and Z-CA4 induced by two-photon absorption at 590 nm in DMSO solution. (A) Power dependence of FI from E-CA4 (0.4 mM) versus laser power at the sample. (B) Percentage conversion of Z-CA4 to the E-isomer versus laser power at the sample, calculated from the measured FI from a solution of Z-CA4 (1 mM) and a series of calibration curves using solutions of E-CA4 at each laser power.

FIG. 12. TRIR spectra of E-CA4 (10 mM) in DCM measured with 266 nm (150 nJ pulse-1) excitation. The upper series shows spectra recorded with pump-to-probe delays between 1 and 3000 ps. The spectra are colour coded up to 20 ps delay. Thereafter the spectra decay. Below the transient spectra are shown the FTIR ground-state spectra of E-CA4 and Z-CA4 in DCM for comparison. Inset: kinetics for formation and decay of the positive bands at 1471 cm-1 (□) and 1490 cm-1 () and the negative band at 1511 cm-1 (▪).

FIG. 13. TRIR spectra of Z-CA4 (10 mM) in DCM measured with 266 nm (150 nJ pulse-1) excitation. The upper series shows spectra recorded with pump-to-probe delays between 1 and 100 ps. Below the transient spectra are shown the FTIR ground-state spectra of E-CA4 and Z-CA4 in DCM for comparison. Inset: kinetics for formation and decay of the negative band at 1512 cm-1.

FIG. 14. Z-E conversion of Z-CA4 in DMSO at 590 nm.

### DETAILED DESCRIPTION

### Isomerisation of Trans-Combretastatins

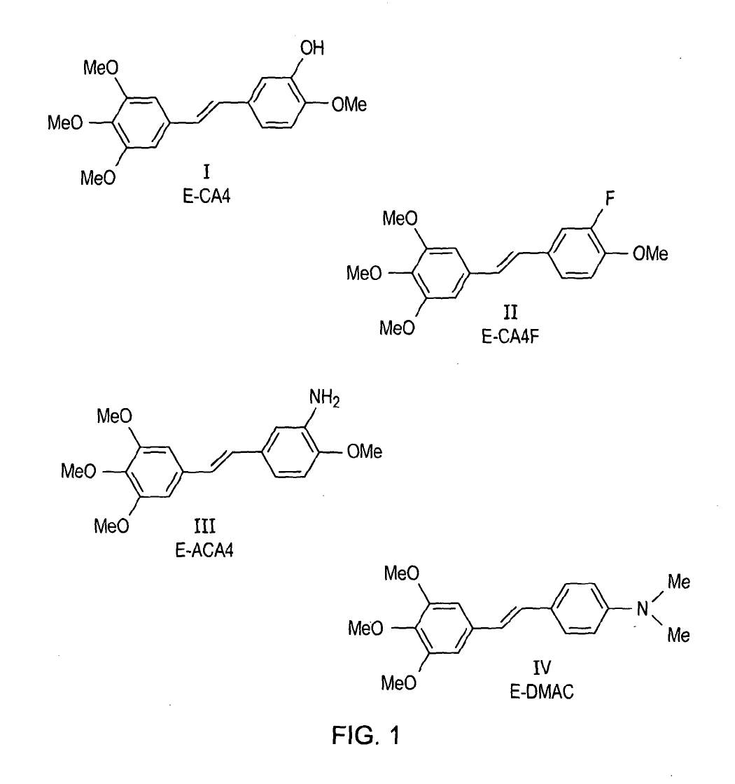

The present invention is based on the real time observation of the intracellular uptake of four E-combretastatins in two types of live cells (HeLa and Chinese hamster ovary (CHO) cells) using fluorescence lifetime imaging (FLIM). The compounds used were the E-isomers of combretastatin A4 (E-CA4) and the analogous active fluorinated derivative (E-CA4F), together with an amino analogue (E-ACA4) and a dimethylamino analogue (E-DMAC) (see FIG. 1). The latter two compounds possessed distinct fluorescence lifetimes and spectra respectively. Also used was E-CNCA4.

The interaction between combrestatins, namely E-CA4F and cultured human umbilical vein endothelial cells (HUVECs) was also studied.

The validity of two photon excitation of E-combretastatins in the wavelength range of 560-630 nm is demonstrated by quantifying fluorescence lifetimes and quantum yields and comparing results with conventional one-photon excitation in the ultraviolet. The information obtained from within the cell using fluorescence intensity images is supplemented by the fluorescence lifetime images which, together with co-localization studies using Nile Red, provide details on the subcellular regions into which the combretastatins accumulate. Furthermore, combinations of intensity and lifetime images allow semi-quantitative measurements of intracellular combretastatin concentrations, demonstrating substantial accumulation of the compounds within both cells types.

Investigation of excited states of E- and Z-CA4 was done by picosecond time-resolved infrared (TRIR) spectroscopy.

The combretastatins normally absorb light in the ultraviolet region, and so would then need to be excited at these wavelengths (300-360 nm) in order to be converted from the less active trans to the more active cis isomer. However light penetration into tissues at these wavelengths is very limited due to competing absorption by many other chromophores. At longer wavelengths (greater than about 620 nm and up to about 900 nm—i.e. in the far red and near infra red (NIR) regions of the spectrum) there is much less absorption and penetration is correspondingly greater. Molecules such as combretastatins may be induced to absorb 2 or more photons in the NIR to provide the same energy as a single photon in the UV—i.e. 2 photons at 640 nm or 3 photons at 960 nm would provide the same energy as one photon at 320 nm. However, while the absorption cross sections vs wavelength for one and two/three photon excitation may not be so simply related, based on the teaching herein, the skilled person would be able to determine appropriate wavelengths for exciting a given combretastatin in a multi-photon process.

In general, in order to promote 2 or 3 photon excitation, high photon densities are required and are readily provided by a laser producing sub-picosecond pulses. Typically a titanium-sapphire laser, e.g. operating the range of 700-1000 nm with a repetition frequency of 80 MHz and a pulse width of ca 150 femtoseconds, may be used and are commercially available. Access to the shorter wavelength region (560-630 nm) may be achieved using an optical parametric oscillator (OPO) laser, driven by the Ti-sapphire laser as described above. Advantageously, the laser beam would need to be focussed and scanned across the area to be illuminated in order to optimise treatment.

In the present invention, the light for the multi-photon isomerisation is provided by a laser capable of providing high photon densities. Broadly speaking, the powers employed are in the range of MW cm⁻² to GW cm⁻² and may be provided in the near infra red by focussing to the diffraction limit the outputs of either a sub-picosecond pulsed titanium sapphire laser (typical average power ca 1-5 mW) or a continuous wave near infra red laser with a power of ca 100 mW. See for example Konig, Multiphoton Microscopy in Life Sciences. Journal of Microscopy 200 (2): 83-104, 2000.

### Pharmaceutical Compositions

As described herein, the present invention may be used in methods of treating cancer or a condition characterised by abnormal vasculature by exploiting the anti-angiogenic properties of the combretastatin family of compounds. Examples of types of cancer for which treatment with combretastatins has be employed include soft tissue, sarcoma, colon cancer, kidney cancer, ovarian cancer, liver cancer, rectal cancer, adrenal cancer, breast cancer, lung cancer, melanoma, oesophageal cancer, osteosarcoma, thyroid cancer, intestinal cancer, acute myeloid leukaemia, myelodysplastic syndrome and hepatic tumours. Generally, combretastatin work by binding to tubulin present in tumour vasculature causing death in the vascular cells of tumours, thereby reducing blood flow and reducing tumour volume. However, the methods of treatment of the present invention may be used in the treatment of other conditions characterised by abnormal vascular proliferation, such as macular degeneration, diabetic retinopathy, endometriosis or psoriasis.

The combretastatin family of molecules that can be used in accordance with the present invention may be administered alone, but it is generally preferable to provide them in pharmaceutical compositions that additionally comprise with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. Examples of components of pharmaceutical compositions are provided in Remington's Pharmaceutical Sciences, 20th Edition, 2000, pub. Lippincott, Williams & Wilkins.

These combretastatins or derivatives thereof may be used in the present invention for the treatment of cancer. As used herein “derivatives” of the therapeutic agents includes salts, coordination complexes, esters such as in vivo hydrolysable esters, free acids or bases, hydrates, prodrugs or lipids, coupling partners.

Salts of the compounds of the invention are preferably physiologically well tolerated and non toxic. Many examples of salts are known to those skilled in the art. Compounds having acidic groups, such as phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g., amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Examples in the prior art of salts or prodrugs of combretastatins focus on forming salts or derivatives at the phenolic hydroxyl group of combretastatin. These include sodium phosphate salts, sodium and potassium salts (U.S. Pat. No. 5,561,122), lithium, caesium, magnesium, calcium, manganese and zinc salts of combretastatins, and ammonium cation salts with imidazole, morpholine, piperazine, piperidine, pyrazole, pyridine, adenosine, cinchonine, glucosamine, quinine, quinidine, tetracycline and verapamil (WO 99/35150).

Esters can be formed between hydroxyl or carboxylic acid groups present in the compound and an appropriate carboxylic acid or alcohol reaction partner, using techniques well known in the art. Salts of the compounds of the invention are preferably physiologically well tolerated and non toxic. Many examples of salts are known to those skilled in the art. Compounds having acidic groups, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art. Examples of esters include those formed between the phenolic hydroxyl of the substituted stilbenes and carboxylic acids, hemisuccinic acid esters, phosphate esters, BOC esters, sulphate esters and selenate esters.

Derivatives which as prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

The term “pharmaceutically acceptable” as used herein includes compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The active agents disclosed herein for the treatment cancer according to the present invention are preferably for administration to an individual in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, Lippincott, Williams & Wilkins. A composition may be administered alone or in combination with other treatments, either simultaneously or sequentially, dependent upon the condition to be treated.

The formulations may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The compounds disclosed herein for the treatment of cancer may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Compositions comprising agents disclosed herein for the treatment of cancer may be used in the methods described herein in combination with other pharmaceutical agents, in particular in combination with other anti-angiogenic agents, with chemotherapeutic agents or in conjunction with radiotherapy. Examples of additional agents that may be employed with the combretastatins in accordance with the present invention include one or more of paclitaxel, carboplatin, and/or doxorubicin.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound, and so the actual weight to be used is increased proportionately.

### Experimental Examples

### Materials and Methods

Solvents for spectroscopy (analytical or spectroscopic grade) were purchased from commercial sources such as Sigma-Aldrich or Alfa Aesar and used as provided by the supplier. For synthesis anhydrous dimethylformamide was purchased from Sigma-Aldrich, whilst tetrahydrofuran and diethyl ether were freshly distilled over sodium and benzophenone under an argon atmosphere as required. Nile Red, 2-aminopyridine (2APY) and L-tryptophan were supplied by Sigma-Aldrich and 9-chloroanthracene (9CLA) from Alfa Aesar.

All combretastatin derivatives investigated (E-CA4, E-CA4F, E-ACA4 and E-DMAC) were synthesized according to published procedures. E-CA4 ((E)-1-(3′,4′,5′-Trimethoxyphenyl)-2-(4″-methoxy-3″-hydroxy-phenyl)ethene) was synthesized using Witting methodology according to Petitt et al. [2]. E-CA4F ((E)-1-(3′,4′,5′-trimethoxyphenyl)-2-(3″-fluoro-4″-methoxyphenyl)ethene) was synthesized using a slightly modified version of the synthesis previously described by Lawrence et al. [3]. E-ACA4 ((E)-1-(3′-Amino-4′-methoxyphenyl)-2-(3″,4″,5″-trimethoxyphenyl)ethene) was prepared via a nitro-derivative [4] followed by reduction to the amine with powdered zinc in glacial acetic acid [5]. E-DMAC ((E)-1-(3′,4′,5′-Trimethoxyphenyl)-2-(4″-dimethylamino-phenyl)ethene) was synthesised as described previously [6]. CNCA4 (4-[2-(3,4,5-trimethoxyphenyl)vinyl]benzonitrile) synthesis [15]. All compounds were purified by flash column chromatography on silica gel (35-70 μm diameter, 60 Å pore size) with petroleum ether:EtOAc 19:1 as solvent, followed by recrystallization from ethanol. Identity and purity of the materials were confirmed by thin layer chromatography and ¹H and ¹³-C NMR spectroscopy at 400 MHz and 100 MHz respectively (Bruker AC-400). Log P values were obtained using Molinspiration software (www.molinspiration.com).

Both HeLa and Chinese hamster ovary (CHO) cells were obtained from the European Collection of Cell Cultures. HeLa cells were cultured in MEM medium (Gibco) containing 10% foetal calf serum, penicillin (100 units/mL), streptomycin (100 μg/mL) and glutamine (2 mM). CHO cells were cultured in DMEM (Gibco) with the same additions as for the HeLa cells. For microscopy the adherent cell cultures were grown in 35 mm diameter glass-bottom culture dishes with optical quality glass (MatTek Corporation) and incubated at 37° C. in a humidified atmosphere containing 5% CO₂. Human umbilical vein endothelial cells (HUVECs) were obtained from TCS Cellworks. HUVECs were sub-cultured in basal medium (TCS Cellworks) supplemented with antibiotic supplement/amphotericin-B, following the detailed guidelines provided by TCS Cellworks.

Absorption spectra were measured in either a Perkin Elmer Lambda 25 or 950 spectrophotometer. Fluorescence measurements were made with either a Spex Fluoromax or Varian Cary Eclipse spectrofluorimeter using the spectral correction curves supplied by the manufacturers. Fluorescence quantum yields with UV (one-photon) excitation were determined using either 2-aminopyridine (Φ_{ref} 0.6 [7]) or 9-chloroanthracene (Φ_{ref} 0.50 in dichloromethane, determined here relative to 9,10-diphenylanthracene in cyclohexane (Φ_{ref} 0.97) and anthracene in ethanol (Φ_{ref} 0.28 [8]) as standards. Fluorescence quantum yields were determined from the relative slopes of plots of integrated fluorescence intensity versus (1-10^{-A}) for samples having absorbance A≦0.1. The square of the refractive index (n²) correction was applied when sample and reference compounds were in different solvents. Irradiation of the samples was minimized by the use of narrow excitation slits to reduce simultaneous photoisomerization. Repeated fluorescence scans of the same sample under these conditions showed almost no change in fluorescence intensity.

The fluorescence lifetime imaging system with multiphoton excitation employed lasers for multiphoton excitation consisting of a Ti:Sapphire laser, tuneable between 700 and 1000 nm and providing ca 180 fs pulses at 76 MHz, and an optical parametric oscillator (OPO) pumped by a second Ti:Sapphire laser source. Using the doubled idler output of the OPO, useful energies (>200 mW) could be obtained between 550 and 640 nm. Samples were excited on the stage of Nikon TE2000U inverted microscope using a water immersion ×60 objective of NA 1.2. Using the various microscope ports it was possible to record the fluorescence in a number of ways. The fluorescence signal at one port was relayed to a spectrometer (Acton 275) and CCD setup (Andor iDUS) so that emission spectra and intensities could be recorded. Alternatively the fluorescence signal could be routed via a second port to a time-correlated single photon counting (TCSPC) system (Becker and Hickl SPC-830) allowing fluorescence lifetime measurements and fluorescence lifetime imaging (FLIM), using Becker and Hickl software (SPCImage) for analysis. In FLIM mode the fluorescence was isolated by a suitable narrowband interference filter (400IU25 together with BG3 (Comar)). The sample could also be imaged using a confocal imaging system (Nikon eC1-Si) installed on a second port of the same microscope using 488 and 543 nm excitation. Cell images were overlaid using Adobe Photoshop CS3. Two photon cross sections were determined using a reference fluorophore as a two photon standard as described by Mathai et al. [9], employing published values of the two-photon cross section for 9-chloroanthracene [10].

The ultrafast time-resolved infrared (TRIR) experiments were performed on the ‘ULTRA’ laser system at the Science and Technology Facilities Council Rutherford Appleton Laboratory. The capabilities of the ULTRA instrument are described in detail elsewhere [16]. Briefly, a chirped pulse amplified titanium:sapphire laser operating at 10 kHz repetition rate produced 40-80 fs duration pulses at a wavelength of 800 nm. The UV excitation pulse was generated from third harmonic generation of the 800 nm output of the amplifier with ˜50 fs duration at 266 nm, irradiating samples with a 1 μJ, 100 μm diameter beam. The mid-IR probe pulse was generated using ˜0.4 mJ of the 800 nm femtosecond output to pump an optical parametric amplifier, and difference frequency mixing of the signal and idler components. The mid-IR probe output pulses had ˜500 cm-1 bandwidth and ˜50 fs pulse duration. The UV pulses were polarized at a magic angle to the IR probe laser polarization. The timing between the pump and probe was performed using a PCcontrolled optical delay line. The sample in solution was placed between two 25 mm diameter CaF2 plates in a rasterscanned Harrick cell to limit irradiation of the sample by multiple pulses. The optical path of the sample between the two windows was controlled using a 100 μm PTFE spacer and the solution flowed using a peristaltic pump. The intensity of the transmitted mid-IR light was dispersed by a grating onto a pair of 128-element mercury-cadmium telluride array detectors (IR Associates), providing full (i.e. ˜500 cm-1) spectral coverage with every laser pulse. The overall instrument response time was ˜200 fs. A reference spectrum of the laser pulse (before the Harrick cell) was accumulated on a third, 64-element array detector and was used to remove the effects of laser fluctuations from the transient absorption data. Ground-state FTIR spectra were recorded using a Nicolet Avatar 360 spectrometer.

### Results and Discussion

### 1. Fluorescence Properties—Spectra, Quantum Yields and Fluorescence Lifetimes

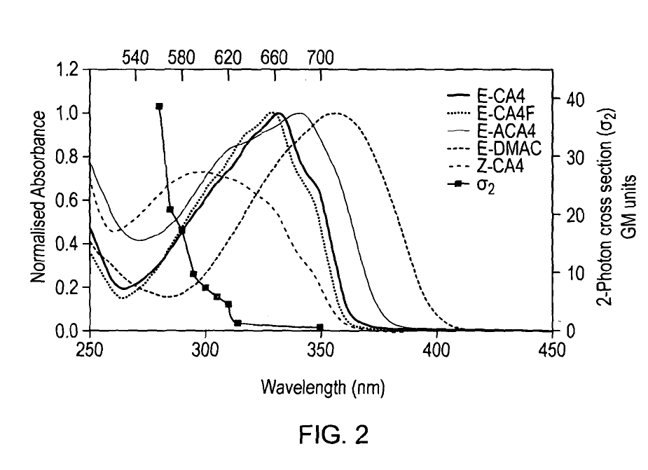

The absorption and fluorescence emission spectra of E-CA4 and E-CA4F in dichloromethane are similar (FIG. 2. E-CA4 and E-CA4F have ultraviolet absorption maxima at 332 and 329 nm respectively, whilst E-ACA (λₘₐₓ 341) and E-DMAC ((AX, 357 nm) absorb at longer wavelength. In contrast, Z-CA4 has a less intense absorption maximum at shorter wavelength (298 nm). The fluorescence quantum yields (Φ) for combretastatins with UV excitation (290-300 nm) were measured using 2-aminopyridine as a reference standard with Φ_{ref} 0.60 [7]. The E-combretastatin quantum yields are strongly dependent on solvent polarity and viscosity and range between 0.05 and 0.4 with the lower values in polar protic solvents (methanol and ethanol) and the higher values in non-polar or viscous polar solvents (hexane and glycerol). The fluorescence quantum yields for E-CA4F were generally somewhat higher than for E-CA4. E-ACA4 displayed higher quantum yields in dichloromethane and acetonitrile than E-CA4 and E-CA4F. In dichloromethane E-DMAC had a quantum yield of 0.11.

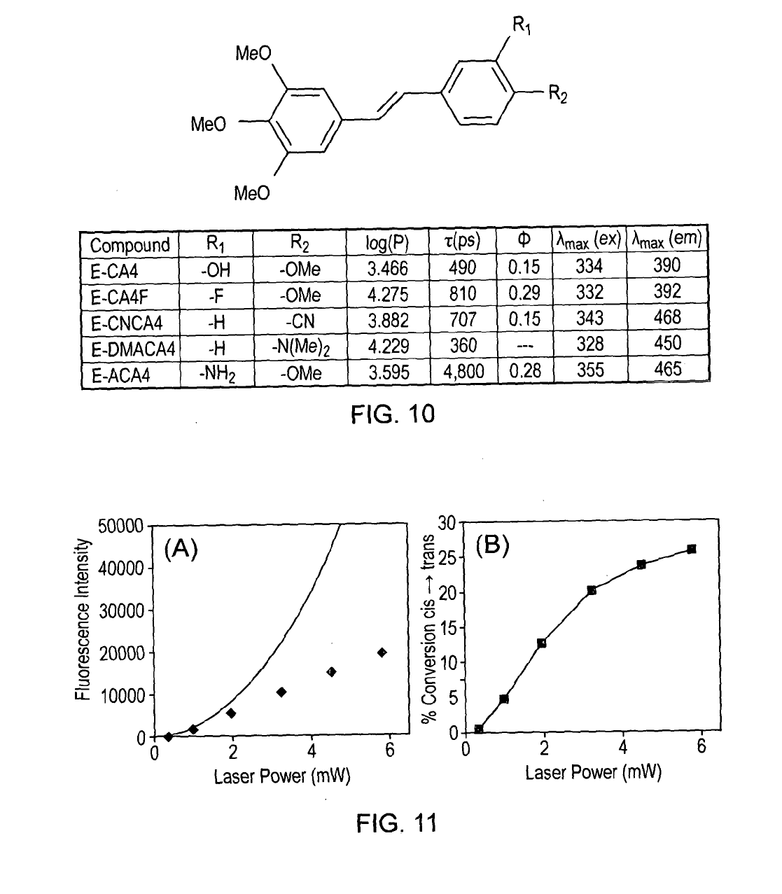

All of the E-combretastatin derivatives shown in FIG. 10 have absorption and fluorescence spectra in the ultraviolet, with typical data for solutions in dimethyl sulfoxide (DMSO) in the figure showing that E-CA4 and E-CA4F have excitation and emission maxima at 332-334 nm and 390-392 nm, respectively, that exhibit little dependence on solvent. The other compounds absorb at similar UV wavelengths but have emission maxima at longer wavelengths in DMSO and are more sensitive to solvent polarity. Fluorescence lifetimes of E-CA4 and E-CA4F were good single exponentials, with that of E-CA4 ranging from 230 ps in methanol through to 1.18 ns in glycerol, an effect due to hindrance by viscosity of the competing isomerization [32].

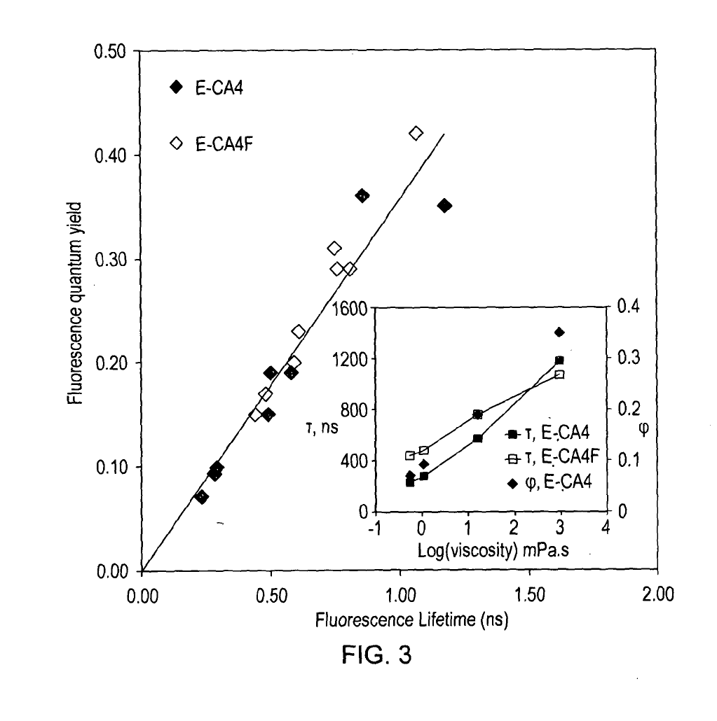

Fluorescence lifetimes were determined using 2PE at 630 nm with the microscope TCSPC setup (overall time response of 25 ps fwhm) and produced the results shown in Table 1. For E-CA4 and E-CA4F all the fluorescence lifetimes were observed to be good exponential decays, whilst both E-ACA4 and E-DMAC exhibited pronounced non-exponential behaviour in many solvents, suggestive of excited charge transfer processes. Characterisation of the spectroscopic properties of these latter two compounds remains to be investigated further. For E-CA4 the lifetimes exhibit a strong solvent dependence, ranging from around 220 ps in polar solvents such as methanol and acetonitrile through to 855 ps in hexane. In the very viscous environment provided by glycerol the lifetime increases further to 1180 ps. In the range of alkanols studied the lifetime is closely correlated with solvent viscosity as shown in the inset to FIG. 3. Consideration of the data from all solvents investigated indicates that the dependence of fluorescence lifetime is a complex function of solvent properties including polarity and viscosity. However it is clear that the measured lifetime has the potential to report on cellular environments in FLIM studies. For E-CA4F similar trends are observed, but the overall differences in both lifetimes and quantum yields over the range of solvents are rather less than for E-CA4. The fluorescence lifetimes of E-CA4F are confined to a narrower range having values ranging from 440 ps in methanol through 740 ps in hexane to 1070 ps in glycerol. The more pronounced solvent dependence for E-CA4 no doubt reflects the more polar nature of the molecule compared with E-CA4F. The relative polarities may be judged from the partition coefficients (P) calculated according to the method of Brown et al. [11]. The respective values of log P for E-CA4 and E-CA4F are 3.81 and 4.62, confirming E-CA4F to be the more lipophilic of the two compounds. A plot of fluorescence quantum yields versus fluorescence lifetime for both E-CA4 and E-CA4F is shown in FIG. 3. The results show a linear relation between intensity or quantum yield (Φ) and lifetime (t) in the form

Φ/Φ₀=t/t₀ (1)

Furthermore both compounds appear to behave similarly indicating they have a similar natural lifetime (τ₀). This is a very useful result with regard to the FLIM microscopy studies described below since it implies that even though the intracellular fluorophore lifetime may vary between (and within) the imaged sample and a calibrating fluorophore solution, the observed intracellular intensities may be adjusted by the use of the lifetime image through equation (1) to obtain quantitative estimates of the intracellular fluorophore concentration.

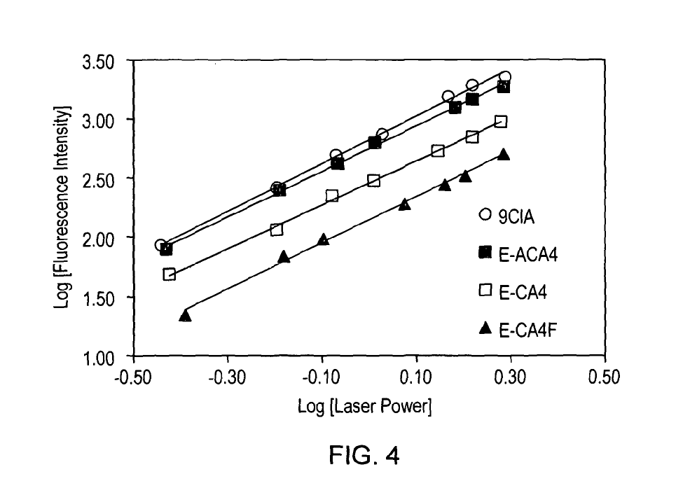

Compared with the commonly used fluorescent labels and probes with absorption and emission maxima in the visible spectral region and with quantum yields approaching unity, these properties of E-CA4 and E-CA4F are considerably less favourable for imaging with fluorescence microscopy. Nonetheless it is possible to excite UV fluorescence in the region of 340-360 nm from intracellular 5-hyroxytryptophan, serotonin and propranolol using 2PE at 630 nm. At this excitation wavelength background interference from intracellular fluorophores such as tryptophan and cofactors is minimised. 2PE excitation of E-CA4, E-CA4, E-ACA4 and E-DMAC at 620 nm is demonstrated by FIG. 4 in which log-log plots of fluorescence intensity (I) versus laser power (P) all demonstrated a slope of 2±0.2, consistent with the anticipated quadratic power dependence for 2PE (equation (2)).

I=σ₂P² (2)

Published cross sections for two photon absorption (σ₂, the probability of an absorption process or photon interaction) typically show large variations depending on the method of measurement, using either the Z-scan method or a reference fluorophore, and the power range and pulse duration of the laser used for excitation [9]. We have attempted to determine values of σ₂ for E-CA4 and E-ACA4 using a reference fluorophore [20]. 9-Chloroanthracene (9CIA) was selected as a standard fluorophore with an appropriate fluorescence spectrum and for which 2PE cross-sections are available [10]. The values of σ₂ at 620 nm for E-CA4 and E-CA4F in DCM were determined to be 2.1±0.5 and 2.75±0.6 GM units respectively. The combination of Ti-sapphire laser and OPO allowed investigation of decreasing wavelength to 560 nm, whereupon E-CA4 showed a substantial increase in cross section (FIG. 2), similar to that reported for bis(diphenylamino)-stilbene by Makarov et al. [10]. Values of σ₂ at 620 nm for E-ACA and E-DMAC were both 5.7±1.2. These larger values may be consistent with some charge transfer character of the excitation of these compounds.

In DMSO solutions, E-CNCA4, E-ACA4 and E-DMACA4 exhibited single exponential decay with lifetimes of 0.71, 4.8 and 0.36 ns, respectively, but showed more complex behaviour in other solvents. In solution, all the compounds in FIG. 10 could be induced to fluoresce by 2PE in the region of 600-630 nm. As usual, this was demonstrated by a quadratic dependence of fluorescence intensity (FI) on laser power (P), i.e. FI CC P². This was also demonstrated for E-combretastatin fluorescence after uptake in live mammalian Cells. 2. Cell Uptake and Intracellular Fluorescence of E-Combretastatins

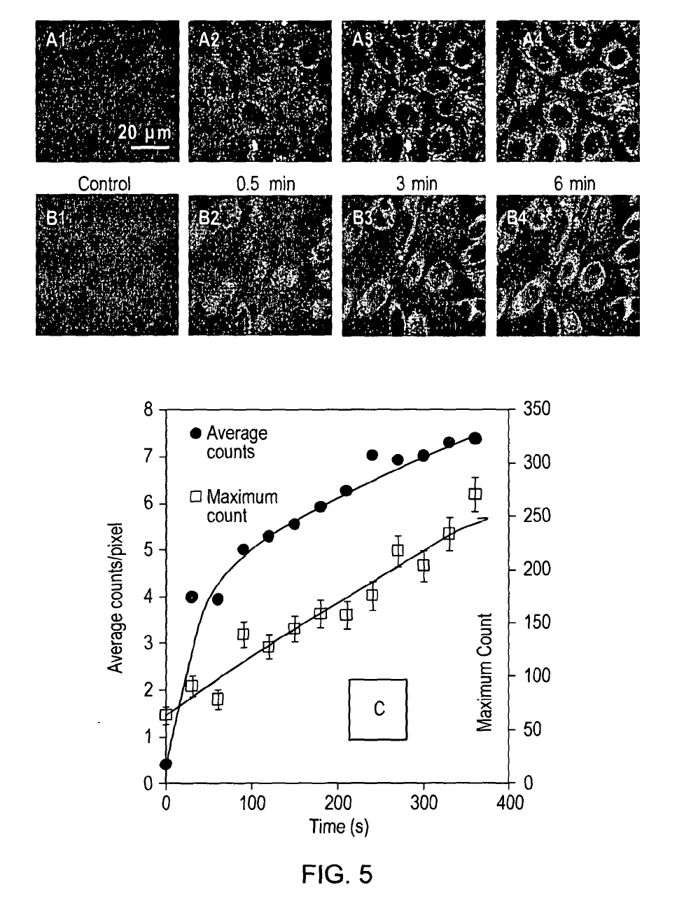

Images of intracellular distribution of E-combretastatins in cell monolayers were obtained using fluorescence lifetime imaging, in which the rastered excitation laser beam dwells on each pixel for a few milliseconds and at each position records a nanosecond fluorescence lifetime decay curve using the time-correlated single photon counting method. The intensity image is constructed from the integrated photon count, which may then be modified using the lifetime information. FIG. 5 shows fluorescence intensity images of CHO cells before and after incubation with either E-CA4 or E-CA4F, using 2PE at 628 nm and measuring fluorescence intensity at 400 nm. The micrographs show clear images of the intracellular distribution of both compounds within the cells minutes after addition. They show very good contrast with cell autofluorescence from the unlabelled cells, which was negligible under the conditions used. This illustrates the advantage of imaging within this spectral window where the excitation wavelength effectively minimises intrinsic fluorescence from tryptophan residues of intracellular proteins and also minimises interference at the emission wavelengths of other cellular fluorophores such as FAD and melanin. The accompanying plots of fluorescence intensity versus time in FIG. 5 show uptake to be biphasic with an initial unresolved rapid phase within the first 100 seconds and a slower subsequent phase lasting over several minutes. The images confirm that both E-CA4 and E-CA4F are excluded from the cell nucleus, and that E-CA4 appears to be located mainly within the cytoplasmic region of the cell. In contrast E-CA4F shows a rather more punctuate distribution in the CHO cells.

HeLa cells were incubated with E-CNCA4 at 37° C. using laser powers (at the sample) of between 0.32 and 0.97 mW. The laser wavelength used for 2PE was 625 nm, providing slightly more energy than at the one-photon absorption maximum at 343 nm. Excellent contrast between E-CNCA4 fluorescence and cell autofluorescence was obtained (data not shown), and for all studies, laser power was limited to 1 mW in order to minimize photodamage to the sample. The plot of the total image FI versus laser power is a good fit to the expected quadratic relationship. For E-CNCA4 in HeLa cells, these FLIM images indicate that the drug accumulates mainly within the cell cytoplasm and is excluded from the cell nucleus.

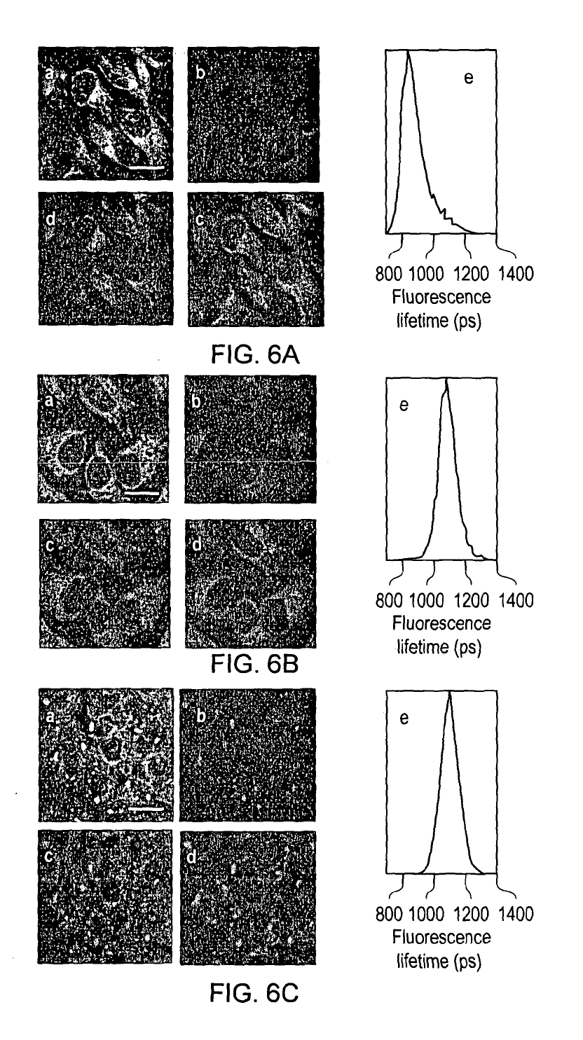

Fluorescence intensity and lifetime images of E-CA4 in HeLa cells are shown in FIG. 6A. The lifetimes range between 750 and 1200 ps, with a peak in the distribution at 900 ps. Similarly E-CA4F in HeLa cells has a peak in the lifetimes' distribution at 1.1 ns (FIG. 6B). A very similar lifetime distribution was measured for E-CA4F in CHO cells (FIG. 6C). These intracellular fluorescence lifetimes are slightly longer than in hexane (0.86 ns, Table1) but similar to that in glycerol (1.18 ns). These relatively long intracellular lifetimes suggest that the combretastatins either occupy a non-polar and/or viscous environment within the cells. The microviscosities of the hydrophobic interiors of lipid bilayer membranes and triglyceride lipid emulsion droplets are between approximately 0.5 and 1 poise and similar to that of glycerol (0.95 poise at 25° C.). Further evidence for the locations of combretastatins in these cells was obtained from labelling with Nile Red, a dye that is known to localise in lipid droplets and membrane systems in cells. Nile Red staining of the cells previously loaded with combretastatins was imaged using the additional confocal scanning facility on the multiphoton microscope using an excitation wavelength of 488 nm to preferentially observe lipid droplets over membranes. Examples of Nile Red stained images are shown in comparison with fluorescence intensity and lifetime images in FIGS. 6A-6C, together with overlaid intensity/lifetime and Nile Red images. FIG. 6C shows the results of E-CA4F uptake in CHO cells. The intensity images show a low background level of fluorescence intensity within the cell cytoplasm and some more intense staining in localised regions that are immediately identified as lipid droplets by co-localisation with the Nile Red stain. Slight discrepancies in the co-localization result from the use of two different imaging systems (the scanned Ti:S laser beam with photon counting for the intensity/lifetime images and the confocal accessory). The higher uptake of E-CA4F into lipid droplets in CHO cells compared with that of E-CA4 (FIGS. 5 and 6) may arise from the higher hydrophobicity of E-CA4F noted above. FIGS. 6B and 6C show uptake of E-CA4 and E-CA4F into HeLa cells. Compared with the CHO cells, there appears to be significantly fewer lipid droplets within the HeLa cells and the compounds are preferentially located within the cytoplasmic regions.

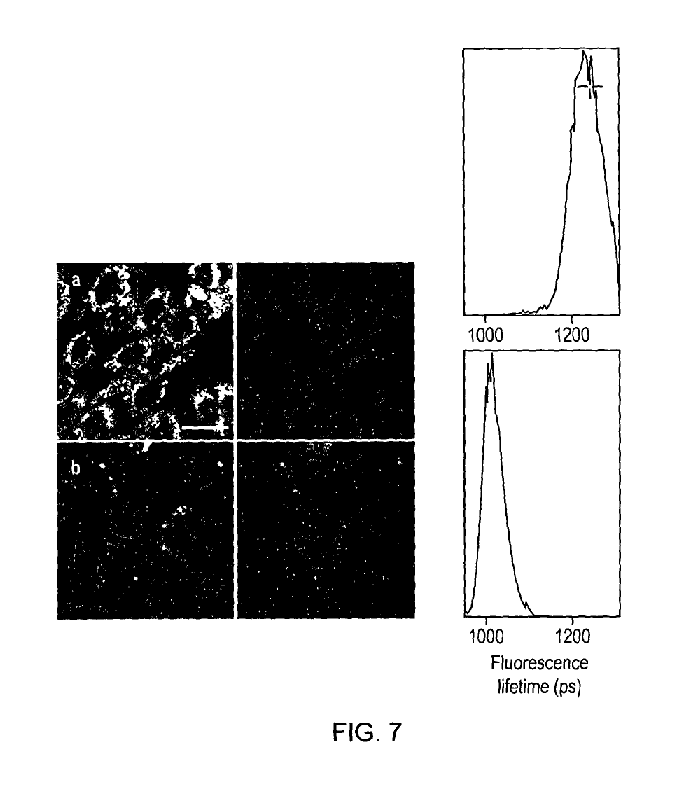

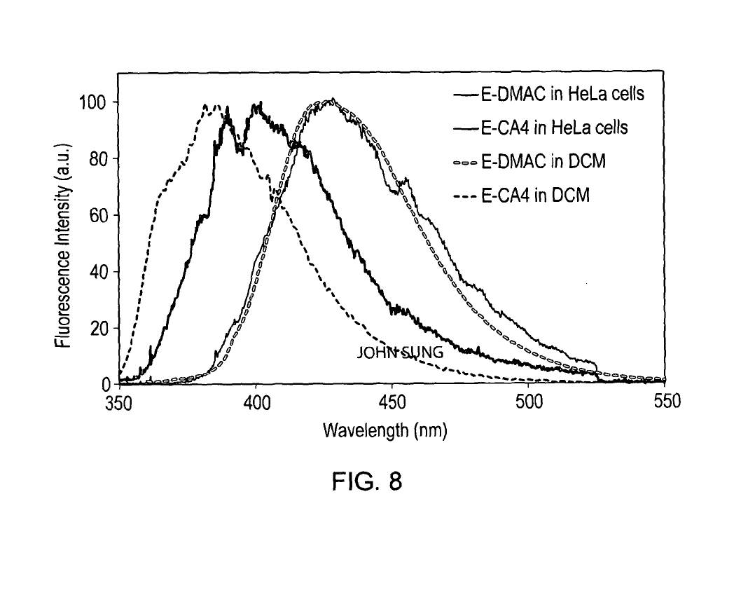

That the fluorescence observed originates from the added combretastatin is confirmed by experiments with E-ACA4 and E-DMAC. FIG. 7 shows fluorescence intensity and lifetime images after uptake of these compounds into CHO cells. The intensity images show a similar cytoplasmic distribution as for E-CA4F, with a few bright regions resembling lipid droplets. This appears consistent with the log P (P is the octanol/water partition coefficient indicative of polarity) for E-DMACA4 being similar to that for E-CA4F (FIG. 10). The similarity with E-CA4F extends to the fluorescence lifetime of E-DMACA4 in DMSO (0.36 ns) which increases to a peak in the lifetime image in CHO cells at 1.07 ns (FIG. 7). For E-ACA4 the log P value is slightly less than for E-CA4F and closer to that for E-CA4. However as a primary amine, it is likely to act as a weak base and protonates to some extent in aqueous solution becoming more polar. Indeed we observe that EACA4 is more soluble in water than the other combretastatins studied here. The fluorescence lifetime is longer than those of the other combretastatins (4.8 ns in DMSO). However the lifetime image for E-ACA4 shows a maximum in the distribution at 2.15 ns, longer than for E-CA4 and E-CA4F. Similarly the fluorescence lifetime of E-ACA4 (1.65 ns) measured for E-ACA4 in hexane (Table 1) is also longer than for E-CA4 and E-CA4F in hexane (0.86 and 0.75 ns respectively). The maximum in the lifetime distribution of E-DMAC in CHO cells is at 1.07 ns and indistinguishable from that of E-CA4F in CHO cells. However the maximum in the fluorescence spectrum of E-DMAC in HeLa cells is clearly at a longer wavelength (FIG. 8) than that for E-CA4 and E-CA4F (FIG. 2). The recorded fluorescence spectrum of E-CA4 in FIG. 8 illustrates the wavelength dependent response of the microscope system in the U, showing a substantial loss in sensitivity below 390 nm. FIG. 8 shows the fluorescence spectra of intracellular combretastatins recorded using the spectrograph and CCD camera attached to the microscope. The spectrum recorded for E-DMACA4 in HeLa cells has a maximum at 430 nm and is very similar to that in solution in dichloromethane, and compares with a peak at 450 nm in the more polar DMSO.

The fluorescence images of E-ACA4 in CHO cells also appear rather different, with FI mainly confined to regions that appear to be intracellular vesicles, reminiscent of the distribution of serotonin in mast cells [17] or propranolol in rat aorta cells [18]. In these instances vesicular uptake is driven by the accumulation of the weak base into acidic compartments in cells such as lysosomes and mitochondria.

Quantification of fluorophore concentration from intensity measurements is often difficult. However this is aided in the present case by the simultaneous recording of fluorescence lifetime images and the observation that fluorescent lifetimes for E-CA4 and E-CA4F are linearly related to quantum yield. Therefore the effect of environment and dynamic quenching of fluorescence, that leads to less radiative decay of the fluorophore, may be taken into account and sample fluorophore concentration (Cₛ) determined quantitatively by comparison of the sample fluorescence intensity (Iₛ) and lifetime (τₛ) with those of a standard reference solution (I_{R}, τ_{R}) of known concentration (C_{R}):—

$${C}_{S} = {I}_{S} ( \frac{{C}_{R}}{{I}_{R}} ) ( \frac{{τ}_{S}}{{τ}_{R}} ) \qquad ( 3 )$$

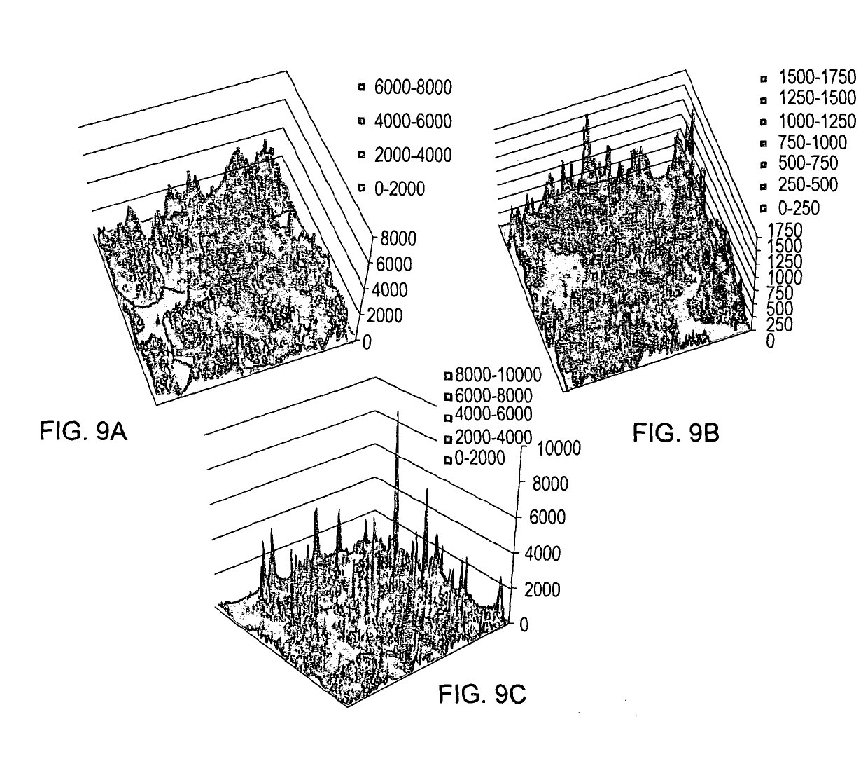

In practice a series of solutions containing increasing concentrations of E-CA4F in DMSO were used to produce a linear calibration curve. DMSO was chosen since it produces a reasonably long lifetime and is more easily handled than glycerol. Using this procedure, concentration maps of intracellular combretastatin were obtained and examples are shown in FIGS. 9A-9C. Initial studies with HeLa cells and E-CA4 added to the serum at 100 μM led to the result in FIG. 9A. Substantial regions of the cell cytoplasm are seen to contain in excess of 2 mM E-CA4 whilst smaller regions contain in excess of 4 mM, or more than 20-40 times the concentration in the medium. This demonstrates that the compound is rapidly taken up and concentrated within the cell, making it available as a drug to interfere with cell function such as microtubule activity. Since the effect of high combretastatin concentration was readily observed, further investigations used only 10 μM combretastatin in the medium: the results of these experiments are shown in FIGS. 9B and 9C. FIG. 9C shows that the concentration of E-CA4F within the lipid droplets in CHO cells, already described above, reaches peaks of almost 10 mM whilst substantial regions of the cells contain around 1 mM of the compound. For the less lipophilic E-CA4 (FIG. 9B) the peak concentrations are not so high but again substantial regions of the cell cytoplasm contain between 250 and 750 μM of the drug. This indicates a concentrating of the drug by a factor of 25-75 times that in the surrounding medium.

3.1. FLIM of E-CA4F Intracellular Distribution in HUVEC Cells and Co-Localization with Nile Red

Whilst studies of intracellular drug uptake in tumour-derived cell lines such as HeLa and CHO were readily observed [19], the mode of action of Z-combretastatins suggests that the true target cells are the endothelial cells that line the blood capillaries [20, 21]. Z-CA4 and analogues exert their anticancer activity by preventing the process of angiogenesis in which a growing tumour is supplied with new vasculature for the supply of nutrients via the bloodstream. We have therefore studied interactions between combretastatins and cultured HUVECs. These non-immortalized cells were found to be more difficult to work with and, unlike most tumour cells, required to be constantly maintained at 37° C. in medium with a CO₂ atmosphere on the microscope stage. Nevertheless it was possible to obtain images of cells after uptake upon E-combretastatin addition to the supporting medium at relatively low concentrations. FI and lifetime images (not shown) of a HUVEC cell after incubation with E-CA4F (5 μM) at 37° C. for 60 min show that the combretastatin is taken up into the cytoplasmic region of the cell, but excluded from the cell nucleus. In addition there are some punctate structures apparent in the images that appear to correlate with lipid droplets identified by Nile Red staining of the same cell. At 37° C. exact co-localization of the sequential images of combretastatin fluorescence and Nile Red labelled lipid droplets, obtained using the two different imaging systems attached to the microscope, is not possible because of the Brownian motion and the occasional rapid saltation of these structures that occur as a result of their transport upon the cellular microtubule network [22]. The fluorescence lifetime distribution of intracellular E-CA4F observed in this cell simultaneously labelled with Nile Red peaks at ˜950 ps. A similar experiment undertaken in the absence of Nile Red (not shown) shows a peak in the lifetime distribution of 1100 ps, indicating lifetime quenching of combretastatin by Nile Red through energy transfer, enabled by weak overlap of the fluorescence spectrum of E-CA4F (λₘₐₓ 390 nm) and the excitation spectrum of Nile Red (λₘₐₓ 450-500 nm) [23]. This confirms that Nile Red and E-CA4F exist in the same space and are able to come close enough to allow FRET. The long lifetime of E-CA4F fluorescence in the HUVEC cell indicates a viscous environment for E-CA4F within the cell which is consistent with a lipidic location such as a membrane or lipid droplet [19]. Since the fluorescence lifetimes and quantum yields of E-CA4F are directly related [19], it is possible to combine the FI and lifetime images to calculate the concentration distribution, using a series of standard solutions (DMSO was the chosen solvent with τ 810 ps). The result shows that intracellular concentrations in many parts of the cell reach 500 μM, or 100 times that of E-CA4F added to the surrounding medium, whilst in the lipid droplets the concentration peaks at over 1 mM. It is therefore clear that E-CA4F (and other compounds in FIG. 10) are taken up by their target endothelial cell and accumulated by up to 200 times the extracellular concentration of the compound. Similar images have been obtained that show corresponding uptake of E-CA4F within HUVECs from solutions containing as little as 1 μM E-CA4F.

3.2. Isomerization of Combretastatin A-4 within the Focal Volume of 2PE

The ultimate objective of this project is to use two-photon activation of a low-activity E-combretastatin to the active Z-isomer with high drug activity within the target site in vivo. At present, experiments are focused on demonstrating effective E-Z interconversion of a combretastatin with red or near-infrared laser 2PE, initially in solution and in cultured cells. There can be no doubt that such isomerization occurs since it originates from the same excited state that produces fluorescence; however, what is at issue is whether such isomerization is sufficiently efficient as to deliver useful amounts of drug capable of exerting a toxic effect on cells and showing a therapeutic effect. Using 2PE the photochemical effects occur within the femtolitre volume of excitation [24]. Although our previous attempts to measure chemical change within a bulk sample have been unsuccessful, the femtolitre volume is significant in comparison with cellular dimensions and converted drug is expected to diffuse from the illuminated femtolitre volume into surrounding regions of the cell. More widely dispersed drug delivery would also be enabled by scanning the beam as in the imaging experiments. At any one instant, the concentration of isomerized compound in the focal volume will represent a steady state achieved through the balance of diffusion from the focal volume and the rate of photochemical formation.

Direct demonstration of the desired E→Z isomerization of CA4 is difficult, as in terms of fluorescence it would involve a loss of FI that may also occur for several reasons. However, an indication that this does occur within solutions is demonstrated in FIG. 11(A) which shows the observed dependence of two-photon excited fluorescence from E-CA4 on laser power up to 6 mW incident on the sample. (This is rather higher power than would normally be used in imaging, when we prefer to use less than 1 mW in order to limit cell damage.) Whilst a good quadratic power dependence (indicated by the solid line) is observed below 1 mW, saturation occurs at higher powers and may at least in part be ascribed to isomerization and the desired formation of Z-CA4. In contrast, the demonstration of E-CA4 as a fluorescent product from 2PE of non-fluorescent Z-CA4 is expected to be much easier to demonstrate.

Calibration curves were constructed of FI versus E-CA4 concentration at each laser power. These were then compared with the FI from the two-photon experiment with Z-CA4 to estimate the concentration of E-CA4 formed within the focal volume. The results in FIG. 11(B) indicate that about 250 μM E-CA4 is formed within the focal volume. This corresponds to the conversion of 25% of the initial concentration of Z-CA4 (1 mM) in solution.

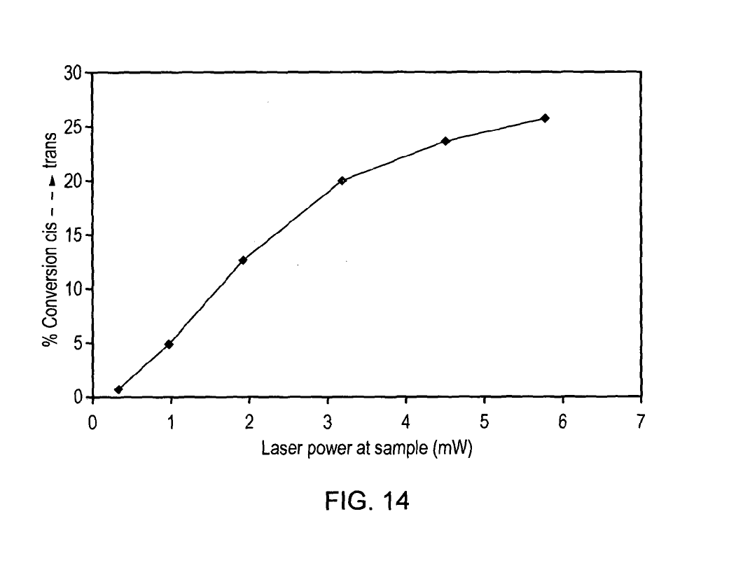

Conversion of Z-CA4 to E-CA4 in DMSO at 590 nm is indicated by formation of fluorescence intensity as power is increased in FIG. 14.

### 3.3. Investigation of Excited States of E- and Z-CA4 by Picosecond TRIR Spectroscopy

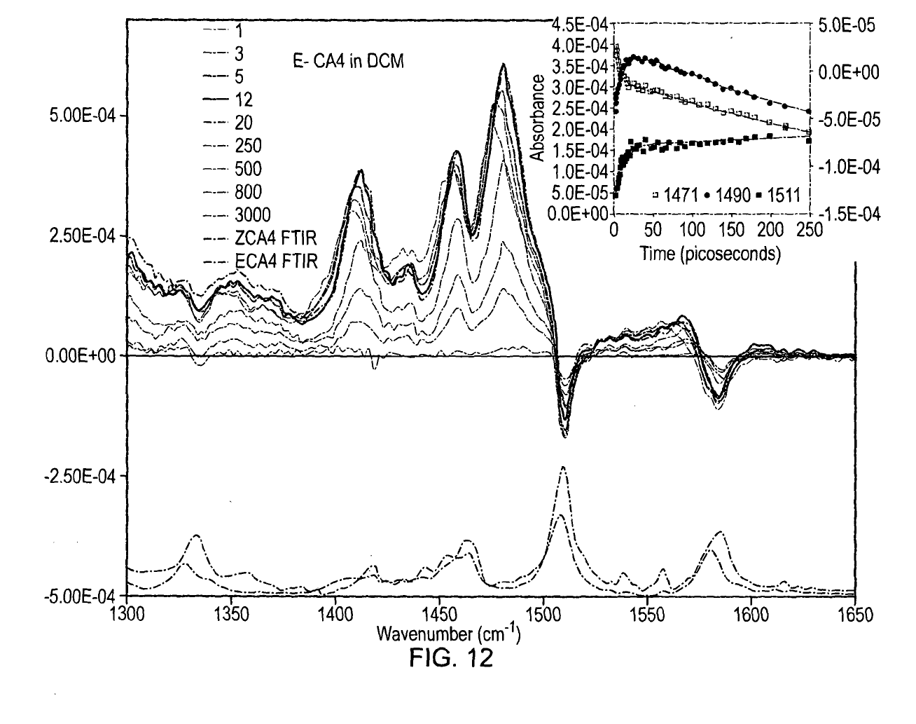

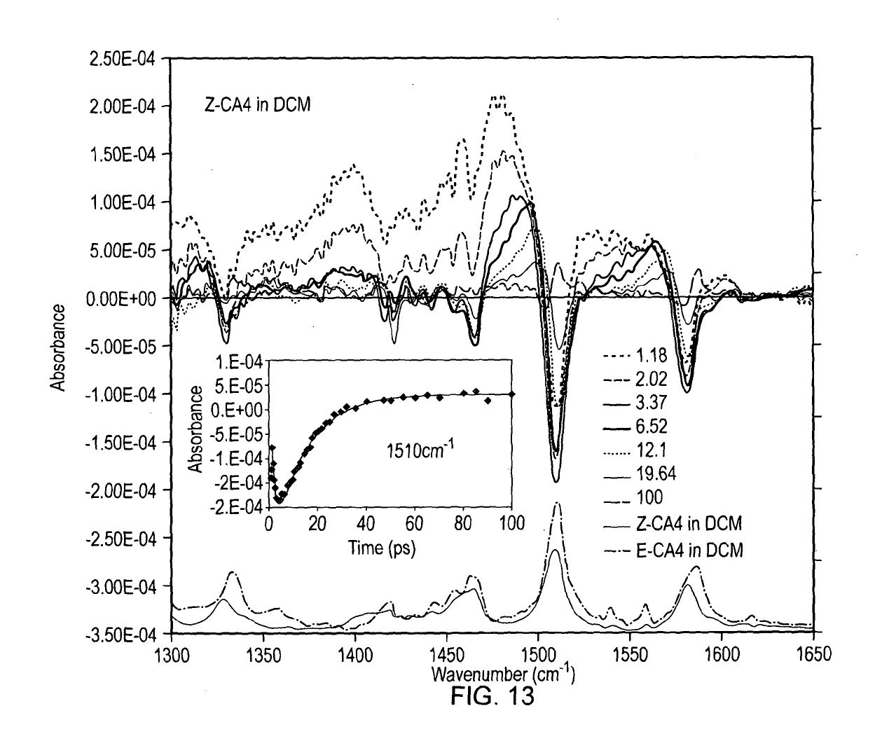

FIGS. 12 and 13 show the TRIR spectra of both isomers of CA4 following ultrafast electronic excitation using 266 nm, with the ground-state FTIR spectra shown beneath the time resolved data for comparison. To date there has been little characterization of combretastatins by infrared spectroscopy [25]. However, the ground-state FTIR spectra have obvious similarities with stilbene and on this basis we assign the 1586/1581 and 1510/1509 cm-1 bands in E-CA4/Z-CA4 to the v8a ring stretches of the phenyl groups and v19a ring stretch with additional contributions from C—H in-plane bending. In the excited state spectra, there are negative absorption bands corresponding to bleaching of the ground-state absorptions and both the E- and Z-isomers are dominated by transient bands between 1350 and 1500 cm-1. At early times the transient bands show rapid shifting over about 10 cm-1 to higher wavenumbers associated with rapid ‘cooling’ of excess vibrational energy following electronic excitation as commonly observed in other molecular systems including DNA [26] and for the C═C stretching band of E-stilbene observed by time-resolved resonance Raman spectroscopy [13]. It is also notable that for E-CA4 at pump-probe delays up to 20 ps, the intensities of the transient bands increase and this is most likely due to molecular rearrangements taking place lowering the overall symmetry of the molecule and increasing the cross-sections of these IR active modes. The converse is true for Z-CA4 and the intensities of the groundstate bleached bands are equal to or greater than those of the excited state transient bands and whilst spectral shifting, as for the E-isomer, occurs due to rapid cooling we do not see any increases in intensity. This indicates that the molecular frame is likely becoming more symmetric in this case.

Furthermore, whilst the ground-state FTIR spectra are similar for both the E- and Z-forms, the TRIR spectra are very different suggesting that the E- and the Z-forms do not share a common intermediate structure. As with stilbene, the symmetry of the molecule in the ground and excited states of both E- and Z-isomers means that the central C═C (double bond) whilst having an extremely high Raman intensity [27] is not observable in the FTIR/TRIR spectra. This is also the case for diphenylacetylene and indeed in their TRIR study, Ishibashi et al [28] observe that in the excited-state TRIR spectrum, the out of phase v8a and v19a modes shift to lower wavenumber from their ground-state positions by 45 and 46 cm-1, respectively. On this basis we tentatively assign the transient bands at 1481 and 1459 cm-1 to correspond to the v8a and v19a modes of Si, respectively. The kinetics of formation and decay of the S1 states of E-CA4 and the Z-CA4 are vastly different and again reflect those of the parent stilbene in so far as the rate of excited state decay of Z-isomer is an order of magnitude faster than for the E-isomer. The kinetics were fitted to a biexponential decay, see insets in FIGS. 12 and 13, for all the transient and recovery (bleach) bands giving average values for E-CA4 in dichloromethane of 6.5±0.46 and 503±56 ps and for Z-CA4 1.5±0.15 and 10.1±0.71 ps. The shorter lifetime is the rapid cascading from S1 (u>1) and the longer lifetime the relaxation time of the equilibrated upper electronic state. This lifetime of the relaxed excited state of E-CA4 measured by TRIR agrees well with the fluorescence lifetime of 500 ps shown in FIG. 10. Furthermore, the lifetime of the relaxed excited state measured by TRIR for E-CA4 in methanol (230±14 ps) is also the same as the fluorescence lifetime in methanol (230 ps). On close inspection of the TRIR spectra for Z-CA4 (FIG. 13), one is able to directly observe the Z-E-isomerization via the small positive growing bands at 1512 and 1586 cm-1.

### CONCLUSIONS

Overall these results show that the E-combretastatins are rapidly (within minutes) taken up by live mammalian cells and reach intracellular concentrations many times those of the medium. In typical toxicity assays the concentrations of Z- and E-combretastatin A4 required to kill these cells are determined to be in the nanomolar and micromolar regions respectively. However the present results suggest that the true intracellular concentrations are much higher and may correspond more closely to the micromolar concentrations at which these compounds are observed to inhibit microtubule assembly. Since the active Z-combretastatins exhibit very weak fluorescence, only with the less active but more fluorescent E-combretastatins has it been possible here to observe the rapid intracellular accumulation. However, it should be noted that this is likely to be a passive process depending mainly on the lipophilicity of these compounds. Since the E- and Z-isomers are likely to differ very little in terms of lipophilicity, it may be expected that Z-combretastatins are similarly accumulated within mammalian cells.

The intracellular concentrations of E-combretastatin derivatives within representative drug target cells (HUVECs) may be estimated by FLIM. The compounds are accumulated within the cell, residing mainly within lipid structures, and reach several hundred times the concentration of that in the extracellular medium. Evidence is provided to demonstrate E→Z isomerization of combretastatin A-4 by two-photon absorption within the focal volume of a near-infrared femtosecond laser beam, suggesting that this may provide an in vivo method of drug activation. Further studies are underway in an attempt to observe the cellular effects of Z-combretatstatins generated intracellularly in this manner. Although two-photon induced PDT involving the intermediacy of singlet oxygen has already been demonstrated [14], we believe that this is the first approach using isomerization of stilbene derivatives. Finally the dynamics of excitation and isomerization have been investigated by ultrafast TRIR spectroscopy, showing initial ultrafast cooling of the initial state and subsequent isomerization, with the excited state of Z-CA4 being much shorter lived than that of the E-isomer.

TABLE 1
Fluorescence lifetimes (in ns) and quantum yields for
combretastatin fluorescence in a range of solvents at 20° C. The
lifetimes were measured. using two-photon excitation. (620-630 nm).
The fluorescence quantum yields were obtained with 290-300 nm
excitation with 2-aminopyridine in H₂SO₄ (0.2 mol dm⁻³) as the
fluorescence standard (0.60). The quantum yields are estimated
to be ±10%. Fluorescence decays were all good exponentials with
estimated lifetimes ±5% except when indicated for E-ACA and E-
DMAC with non-exponential (“ne”) behaviour observed as indicated.

| | E-CA4 | | E-CA4F | | E-ACA | | E-DMAC | |
|---|---|---|---|---|---|---|---|---|
| | τ | φ | τ | φ | τ | φ | τ | φ |
| MeOH | 0.23 | 0.071 | 0.44 | 0.15 | ne | 0.11 | ne | — |
| EtOH | 0.28 | 0.093 | 0.48 | 0.17 | ne | — | ne | — |
| Ethylene glycol | 0.58 | 0.19 | 0.76 | 0.29 | ne | — | ne | — |
| Glycerol | 1.18 | 0.35 | 1.07 | 0.42 | ne | — | — | — |
| MeCN | 0.29 | 0.099 | 0.59 | 0.2 | 3.6 | 0.41 | ne | — |
| DCM | 0.50 | 0.19 | 0.61 | 0.23 | — | 0.43 | ne | 0.11 |
| DMSO | 0.49 | 0.15 | 0.81 | 0.29 | 4.8 | 0.28 | 0.36 | — |
| Hexane | 0.86 | 0.36 | 0.75 | 0.31 | 1.65 | 0.38 | 0.41 | — |

### REFERENCES

The documents disclosed herein are all expressly incorporated by reference in their entirety.
1. Pettit et al, (1982) Anti-neoplastic agents. 84. Isolation and structure of combretastatin. Can. J. Chem. 60, 1374-1376.
2. Pettit et al, (1995) Antineoplastic Agents. 291. Isolation and synthesis of combretastatins A-4, A-5, and A-6. J. Med. Chem. 38, 1666-1672.
3. Lawrence et al, (2003) Synthesis and anticancer activity of fluorinated analogues of combretastatin A-4. J. Fluorine Chem. 123, 101-108.
4. Pinney et al, (2000) Synthesis and biological evaluation of aryl azide derivatives of combretastatin A-4 as molecular probes for tubulin. Bioorgan. Med. Chem. 8, 2417-2425.
5. Hirst, “Targeting the Colchicine Binding Site of Tubulin with Combretatstatin Derivatives,” UMIST, Manchester, PhD Thesis 2004.
6. Cushman et al, (1991) Synthesis and evaluation of stilbene and dihydrostilbene derivatives as potential anticancer agents that inhibit tubulin polymerization. J. Med. Chem. 34, 2579-2588
7. Rusakomicz et al, (1968) 2-Aminopyridine as a standard for low-wavelength spectrofluorimetry. J. Phys. Chem. 72, 2680-2681.
8. Suzuki et al, (2009) Reevaluation of absolute luminescence quantum yields of standard solutions using a spectrometer with an integrating sphere and a back-thinned CCD detector. Phys. Chem. Chem. Phys. 11, 9850-9860.
9. Mathai et al, (2007) Two-photon absorption cross sections and time-resolved imaging using phorphyrin photosensitisers. Photochem. Photobiol. Sci. 6, 1019-1026.
10. Makarov et al, (2008) Two-photon absorption standards in the 550-1600 nm excitation wavelength range. Opt. Express 16, 4029-4047.
11. Brown et al, (2000) Comparative molecular field analysis of colchicine inhibition and tubulin polymerization for combretastatins binding to the colchicine binding site on R-tubulin. Bioorgan. Med. Chem. 8, 1433-1441
12. Lawrence N J, Rennison D, Woo M, McGown A T and Hadfield J A 2001 Antimitotic and cell growth inhibitory properties of combretastatin A-4-like ethers Bioorg. Med. Chem. Lett. 11 51-4
13. Iwata K and Hamaguchi H 1997 Microscopic mechanism of solute-solvent energy dissipation probed by picosecond time-resolved Raman spectroscopy J. Phys. Chem. A 101 632-7
14. Dahlstedt E, Collins H A, Balaz M, Kuimova M K, Khurana M, Wilson B C, Phillips D and Anderson H L 2009 One- and two-photon activated phototoxicity of conjugated porphyrin dimers with high two-photon absorption cross sections Org. Biomol. Chem. 7 897-904
15. Osterod F, Peters L, Kraft A, Sano T, Morrison J J, Feeder N and Holmes A B 2001 Luminescent supramolecular assemblies based on hydrogen-bonded complexes of stilbenecarboxylic acids and dithienol[3,2-b:2′,3′-d]thionene-2-carboxylic acids with a tris(imidazoline) base J. Mater. Chem. 11 1625-1533
16. Greetham G M et al 2010 ULTRA: a unique instrument for time-resolved spectroscopy Appl. Spectrosc. 64 1311-9
17. Williams R M, Shear J B, Zipfel W R, Maiti S and Webb W W 1999 Mucosal mast cell secretion processes imaged using three-photon microscopy of 5-hydroxytryptamine autofluorescence Biophys. J. 76 1835-46
18. Bisby R H, Crisostomo A G, Botchway S W, Parker A W and Watts S W 2009 Intracellular imaging of serotonin and propranolol in rat aorta cells Central Laser Facility Annual Report 2008/2009 RAL-TR-2009-025 (ISBN 978-0-9556616-5-5) pp 150-2
19. Bisby R H, Botchway S W, Hadfield J A, McGown A T, Parker A W and Scherer K M 2011 Fluorescence lifetime imaging of E-combretastatin uptake and distribution in live mammalian cells Eur. J. Cancer Online, doi:10.1016/j.ejca.2011.11.025
20. Dark G G, Hill S A, Prise V E, Tozer G M, Pettit G R and Chaplin D J 1997 Combretastatin A-4, an agent that displays potent and selective toxicity toward tumor vasculature Cancer Res. 57 1829-34
21. Hotchkiss K A, Ashton A W, Mahmood R, Russell R G, Sparano J A and Schwartz E L 2002 Inhibition of endothelial cell function in vitro and angiogenesis in vivo by docetaxel (taxotere): association with impaired repositioning of the microtubule organizing center Mol. Cancer Ther. 1 1191-200
22. Digel M, Ehehalt R and Fullekrug J 2010 Lipid droplets lighting up: insights from live microscopy FEBS Lett. 584 2168-75
23. Greenspan P, Mayer E P and Fowler S D 1985 Nile red: a selective fluorescent stain for intracellular lipid droplets J. Cell Biol. 100 965-73
24. Botchway S W, Crisostomo A G, Parker A W and Bisby R H 2007 Near infrared multiphoton-induced generation and detection of hydroxyl radicals in a biochemical system Arch. Biochem. Biophys. 464 314-21
25. Padmaja L, Ravikumar C, Sajan D, Joe I H, Jayakumar V S, Pettit G R and Nielsen O F 2009 Density functional study on the structural conformations and intramolecular charge transfer from the vibrational spectra of the anticancer drug combretastatin-A2 J. Raman Spectrosc. 40 419-28
26. Towrie M, Doorley G W, George M W, Parker A W, Quinn S J and Kelly J M 2009 ps-TRIR covers all the bases-recent advances in the use of transient IR for the detection of short-lived species in nucleic acids Analyst 134 1265-73
27. Matousek P, Gaborel G, Parker A, Phillips D, Scholes G, Toner W and Towrie M 1999 PS-TR3 spectroscopy of S1 cis-stilbene in solution Laser Chem. 19 97-100
28. Ishibashi T, Okamoto H and Hamaguchi H 2000 Picosecond transient infrared spectra and structure of S1 diphenylacetylene in solution Chem. Phys. Lett. 325 212-8

1. A compound which is a trans-combretastatin, or a derivative, salt, solvate and/or chemically protected form thereof, for use in a method of treating a cancer or a condition characterised by abnormal vasculature, wherein the method comprises administering the compound to a patient having cancer or a condition characterised by abnormal vasculature and irradiating the compound with light at a wavelength to isomerise the compound in situ from the trans-isomer to the cis-isomer by a multi-photon process.

2. The compound for use in a method of treating cancer or a condition characterised by abnormal vasculature according to claim 1, wherein the trans-combretastatin is represented by Formula I: 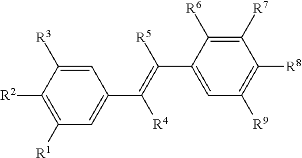
wherein:
R¹, R² and R³ are independently selected from hydrogen, C₁₋₆ alkyl or C₁₋₆ alkoxy, wherein at least one, preferably two, and more preferably all three of R¹, R² and R³ are independently selected from C₁₋₆ alkyl or C₁₋₆ alkoxy;
R⁴ and R⁵ are independently selected from hydrogen, C₁₋₆ alkyl, C₁₋₆ alkoxy, —CO₂H, —CO₂R, —CN, R—C(O)— or —CHO;
R⁶ is selected from hydrogen, hydroxyl, halogen, C₁₋₆ alkyl, C₁₋₆ haloalkyl, C₁₋₆ alkoxy, —O—P(O)(OM⁺)₂ where M+ is a monovalent metal ion, such as sodium or potassium, —NH₂, —NHR, —NRR′, —SR, —NO₂, —CN or —CHO, wherein R and R′ are defined below;
R⁷ is selected from hydrogen, hydroxyl, halogen, —O-ester, —OCO-aryl, —OCO-heteroaryl, —OCO-amino acid, —OCO-peptide, —OCO-polymer, —OCO-sugar, OCO—CHR—NH—BOC, wherein BOC represents a t-butoxycarbonyl group, —O—P(O)(OM⁺)₂ where M+ is a monovalent metal ion such as sodium or potassium, pyridyl ester, —OR, —NO₂, —NH₂, —NHR, —NRR′, —SR, —NHC═OR, —CN or —CHO;
R⁸ is selected from halogen, C₁₋₆ alkyl, C₁₋₆ alkoxy, —NH₂, —NHR, —NRR′, —SR, —CF₃, —CHO, —CN or —C═OR; and
R⁹ is selected from hydrogen, hydroxyl, halogen, C₁₋₆ alkyl, C₁₋₆ haloalkyl, C₁₋₆ alkoxy, hydroxyl, —NH₂, —NHR, —NRR′, —SR, —NO₂, —NH₂, —NHR, —NRR′, —SR, —NHC═OR, —CN or —CHO;
wherein the R and/or R′ substituents, when present, are independently selected from an optionally substituted C₁₋₆ alkyl group;
and salts, solvates, and/or chemically protected forms thereof.

3. The compound for use in a method of treating cancer or a condition characterised by abnormal vasculature according to claim 2, wherein:
R¹, R² and R³ are independently selected from C₁₋₆ alkyl or C₁₋₆ alkoxy; and/or
R⁴ and R⁵ are independently selected from hydrogen or C₁₋₆ alkyl; and/or
R⁶ is hydrogen; and/or
R⁷ is selected from hydrogen, hydroxyl, halogen, —NO₂, or —NH₂; and/or
R⁸ is selected from C₁₋₆ alkoxy or —N(C₁₋₆ alkyl)₂; and/or
R⁹ is selected from hydrogen, hydroxyl, halogen, —NH₂ or —NO₂;
and salts, solvates, and/or chemically protected forms thereof.

4. The compound for use in a method of treating cancer or a condition characterised by abnormal vasculature according to claim 2, wherein a R⁷ and/or R⁹ halogen substituent is fluorine.

5. The compound for use in a method of treating cancer or a condition characterised by abnormal vasculature according to claim 2, wherein C₁₋₆ alkyl or C₁₋₆ alkoxy groups are methyl or methoxy.

6. The compound for use in a method of treating cancer or a condition characterised by abnormal vasculature according to claim 2, wherein the compound represented by Formula I has the following substituents:
R¹, R² and R³ are methoxy, R⁴, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴, R⁵ and R⁶ are hydrogen, R⁷ is fluorine, R⁸ is methoxy, and R⁹ is hydrogen, and salts, solvates, and/or chemically protected forms thereof; or
R¹, R² and R³ are methoxy, R⁴, R⁵, R⁶ and R⁷ are hydrogen, R⁸ is m-CN, and R⁹ is hydrogen, and salts, solvates, and/or chemically protected forms thereof; or
R¹, R² and R³ are methoxy, R⁴, R⁵ and R⁶ are hydrogen, R⁷ is or —NH₂, R⁸ is methoxy, and R⁹ is hydrogen, and salts, solvates, and/or chemically protected forms thereof; or
R¹, R² and R³ are methoxy, R⁴, R⁵ and R⁶ are hydrogen, R⁷ is hydrogen, R⁸ is —N(methyl)₂, and R⁹ is hydrogen, and salts, solvates, and/or chemically protected forms thereof; or
R¹, R² and R³ are methoxy, R⁴, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴ is C₁₋₆ alkyl, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴ is —CN, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴ is —CHO, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴ is —COMe, R⁵ and R⁶ are hydrogen, R⁷ is hydroxyl, R⁸ is methoxy, and R⁹ is hydrogen, and salts, solvates, and/or chemically protected forms thereof; or
R¹, R² and R³ are methoxy, R⁴, R⁵ and R⁶ are hydrogen, R⁷ is —O—P(O)(OM⁺)₂ where M+ is a monovalent metal ion, such as sodium or potassium, R⁸ is methoxy, and R⁹ is hydrogen, and salts, solvates, and/or chemically protected forms thereof; or
R¹, R² and R³ are methoxy, R⁴ and R⁵ are hydrogen, R⁶ and R⁷ are hydroxyl, R⁸ is methoxy, and R⁹ is hydrogen, and salts, solvates, and/or chemically protected forms thereof; or
R¹, R² and R³ are methoxy, R⁴ and R⁵ are hydrogen, R⁶ and R⁷ is —O—P(O)(OM⁺)₂ where M+ is a monovalent metal ion, such as sodium or potassium, R⁸ is methoxy, and R⁹ is hydrogen, and salts, solvates, and/or chemically protected forms thereof.

7. The compound for use in a method of treating cancer or a condition characterised by abnormal vasculature according to claim 2, wherein the light for isomerising the compound in the multi-photon process comprises far red and/or near infra red (NIR) radiation.

8. The compound for use in a method of treating cancer or a condition characterised by abnormal vasculature according to claim 2, wherein the combretastatin is induced to absorb 2 or more photons in the NIR.

9. The compound for use in a method of treating cancer or a condition characterised by abnormal vasculature according to claim 8, wherein 2 photons at a wavelength between 560 nm and 630 nm, preferably about 600 nm.

10. The compound for use in a method of treating cancer or a condition characterised by abnormal vasculature according to claim 9, wherein 3 photons at a wavelength between 840 nm and 1200 nm, preferably about 900 nm.

11. The compound for use in a method of treating cancer or a condition characterised by abnormal vasculature according to claim 2, wherein the light for the multi-photon isomerisation is provided by a laser capable of providing high photon densities in the range of MW cm⁻² to GW cm⁻².

12. The compound for use in a method of treating cancer or a condition characterised by abnormal vasculature according to claim 2, wherein the high photon density is in the near infra red by focussing to the diffraction limit the outputs of either a sub-picosecond pulsed titanium sapphire laser or a continuous wave near infra red laser.

13. The compound for use in a method of treating cancer or a condition characterised by abnormal vasculature according to claim 12, wherein the laser is a titanium-sapphire laser operating the range of 700-1000 nm or the laser is an optical parametric oscillator (OPO) laser, driven by the Ti-sapphire laser providing shorter wavelength region in the range of 560-630 nm.

14. The compound for use in a method of cancer or a condition characterised by abnormal vasculature according to claim 2, wherein method comprises focussing and scanning a laser beam across the area to be illuminated in order to optimise treatment.

15. The compound for use in a method of treating cancer or a condition characterised by abnormal vasculature according to claim 2, wherein the multi-photon process is a two-photon process or a three-photon process.

16. The compound for use in a method of treating cancer or a condition characterised by abnormal vasculature according to claim 2, wherein the cis-isomer has at least three times more cytotoxic to the cancer cells than the trans-isomer, and optionally has at least five times more cytotoxic to the cancer cells than the trans-isomer.

17. The compound for use in a method of treating cancer or a condition characterised by abnormal vasculature according to claim 2, wherein the condition is cancer.

18. The compound for use in a method of treating cancer or a condition characterised by abnormal vasculature according to claim 17, wherein the cancer is a solid tumour.

19. The compound for use in a method of treating cancer or a condition characterised by abnormal vasculature according to claim 17, wherein the method further comprises the step of allowing the compound to localise at the site of a tumour.

20. The compound for use in a method of treating cancer or a condition characterised by abnormal vasculature according to claim 17, wherein the method further comprises the step of targeting the compound to the cancer cells or the site of a tumour in the patient.

21. The compound for use in a method of treating cancer or a condition characterised by abnormal vasculature according to claim 17, wherein the cancer is soft tissue, sarcoma, colon cancer, kidney cancer, ovarian cancer, liver cancer, rectal cancer, adrenal cancer, breast cancer, lung cancer, melanoma, oesophageal cancer, osteosarcoma, thyroid cancer, intestinal cancer, acute myeloid leukaemia, myelodysplastic syndrome and hepatic tumours.

22. The compound for use in a method of treating cancer or a condition characterised by abnormal vasculature according to claim 2, wherein the method comprises treating the patient with the in combination with other pharmaceutical agents, in particular a second anti-angiogenic agent, a second chemotherapeutic agent or in conjunction with radiotherapy.

23. The compound for use in a method of treating cancer or a condition characterised by abnormal vasculature according to claim 2, wherein the condition characterised by abnormal vasculature is macular degeneration, diabetic retinopathy, endometriosis and psoriasis.

24. A kit for use in a method of treating cancer or a condition characterised by abnormal vasculature in a patient, the kit comprising a compound as defined in claim 1 and instructions for irradiating the compound with light at a wavelength to isomerise the compound in situ from the trans-isomer to the cis-isomer by a multi-photon process.

25. A method of treating a cancer or a condition characterized by abnormal vasculature, wherein the method comprises administering to a patient having cancer or a condition characterized by abnormal vasculature a compound which is a trans-combretastatin, or a derivative, salt, solvate and/or chemically protected form thereof, and irradiating the compound with light at a wavelength effective to isomerise the compound in situ from the trans-isomer to the cis-isomer by a multi-photon process.
